(19) United States
(12) Patent Application Publication
(10) Pub. No.: US 2008/0051367 A1
(43) Pub. Date: Feb. 28, 2008

(54) Hyaluronan used in improvement of anti-oxidation and proliferation in chondrocytes

(76) Inventors: Huan-Ching Hsu, Taipei (TW); Ching-Chuan Jiang, Taipei (TW); Chiang-Ting Chien, Taipei (TW)

Correspondence Address: TROXELL LAW OFFICE PLLC, SUITE 1404, 5205 LEESBURG PIKE, FALLS CHURCH, VA, 22041, US

(21) Appl. No.: 11/508,245

(22) Filed: Aug. 23, 2006

(51) Int. Cl. A61K 31/728

(52) U.S. Cl. 514/54

(57) ABSTRACT

The present invention relates to the use of hyaluronan in protecting chondrocytes against oxidative damages and further promoting their proliferation. Particularly, the present invention relates to the use of hyaluronan with a molecular weight of 600,000-1,200,000 Dalton in protecting chondrocytes against reactive oxygen damages and further promoting their proliferation.

### FIELD OF THE INVENTION

The present invention relates to the use of hyaluronan in protecting chondrocytes against oxidative damages and further promoting their proliferation. The present invention relates to the use of hyaluronan in manufacturing medicaments for treating or preventing arthritis and healthy foods for improving the condition of arthritis.

### BACKGROUND OF THE INVENTION

A joint (articulation) is the location at which two bones make contact (articulate). Joints are constructed to both allow movement and provide mechanical support. The surfaces of the two bones at the joint are covered in cartilage. The thickness of the cartilage varies with each joint, and sometimes may be of uneven thickness. Articular cartilage is multi-layered. A thin superficial layer provides a smooth surface for the two bones to slide against each other. Articular cartilage is a complex, living tissue that lines the bony surface of joints. It's function is to provide a low friction surface enabling the joint to withstand weight bearing through the range of motion needed to perform activities of daily living as well as athletic endeavors. Those daily activities include walking, stair climbing, and work-related activities. In other words, articular cartilage is a very thin shock absorber. It is organized into five distinct layers, with each layer having structural and biochemical differences.

Chondrocytes lie in a cavity called a lacuna. Chondrocytes make up “cell nests,” groups of chondrocytes within lacunae. Chondroblasts are responsible for the secretion and maintenance of the matrix. Chondroblasts encased in matrix develop into chondrocytes. The matrix immediately surrounding the chondrocytes is referred to as the territorial matrix and stains darker than the interstitial matrix.

Synovial fluid is a thick, stringy fluid found in the cavities of synovial joints. Synovial fluid reduces friction between the articular cartilage and other tissues in joints to lubricate and cushion them during movement. Normal synovial fluid contains 0.15% (w/v) of hyaluronan (hyaluronic acid), a polymer of disaccharides composed of D-glucuronic acid and D-N-acetylglucosamine joined by alternating beta-1,4 and beta-1,3 glycosidic bonds. Hyaluronan is synthesized by the synoviocytes and secreted into the joint cavity to: (1) increase the viscosity and elasticity of articular cartilages and lubricate the surfaces between synovium and cartilage (see, for example, Pathophysiology. 2003, 9:215-220); (2) combine with certain glucosamines, such as glucosaminoglycan, (GAG) and the like, to form proteoglycan as a main component of the extracellular matrix of chondrocytes; (3) provide a cytoskeleton for chondrocyte in cartilage; and (4) be destructible to oxidative materials (see, Arthritis & Rheumatism. 2003, 8:3151-3158).

Osteoarthritis (OA, also known as degenerative arthritis or degenerative joint disease), is a condition in which low-grade inflammation results in pain in the joints, caused by wearing of the cartilage that covers and acts as a cushion inside joints. As the bone surfaces become less well protected by cartilage, the patient experiences pain upon weight bearing, including walking and standing. Due to decreased movement because of the pain, regional muscles may atrophy, and ligaments may become more lax. OA is the most common form of arthritis. As a person ages, the water content of the cartilage decreases, and the protein composition in it degenerates, thus degenerating the cartilage through repetitive use or misuse.

In chemistry, radicals (often referred to as free radicals) are atomic or molecular species with unpaired electrons on an otherwise open shell configuration. These unpaired electrons are usually highly reactive, so radicals are likely to take part in chemical reactions. Reactive oxygen species (ROS) are species such as superoxide, hydrogen peroxide, and hydroxyl radical and are associated with cell damage. According to the Free Radical Theory of Aging, aging occurs (via a loss of energy prodicing cells) either when mitochondria begin to die out because of free radical damage or, when less functional mitochondria remain within these cells.

Free radicals may be produced in many cellular organelles, such as mitochondria, lysosome, cell membrane, endoplasmic reticulum (ER). The over-production of oxidative materials results in the damages on lipids, proteins or DNA, and further causes diseases or conditions such as cancer, Parkinson's disease, hypertension, atherosclerosis, myocardial infraction, thrombosis, and thromboagglutination. As known that free radicals attact to the unsaturated bindings on lipid bilayer, then process a lipid peroxidation and destroy the cell membrane. Free radicals also disrupt the structure of S-containing proteins, which leads damages to ion transport system and celluar proteins. Additionally, free radicals make breakage and mutation on genetic material, such as DNA, change cell cycle, and even cause cancers.

Appropriately, the first medical application of hyaluronan for humans was as a vitreous supplement/replacement during eye surgery in the late 1950s. The hyaluronan used was isolated initially from human umbilical cord, and shortly thereafter from rooster combs in a highly purified and high molecular weight form. This latter preparation, now sold under the trade name of Healon (Pharmacia), is currently widely used for ophthalmic viscosurgery and in other forms of surgery, as is Opegan (Seikagaku), a hyaluronan product also prepared from rooster comb. Another hyaluronan product, Artz (Seikagaku), was developed for use as a supplement in the synovium of osteoarthritic joints, and a covalently cross-linked form of hyaluronan, Synvisc (Biomatrix), with more pronounced viscoelastic properties, is also being used for the same purpose.

It is known that reactive oxygen, such as OH⁻, H₂O₂, HOCl and the like, will depolarize and destroy the structure of hyaluronan in arthritis pathology. Reactive oxygen further reduces the level of hyaluronan, decreases the viscosity and lubrication in joint, and induces inflammatory reaction (see, for example, Free Radic Biol Med. 2003, 35:169-78; and Pathophysiology. 2003, 9:215-220). The structure of hyaluronan with high MW (such as 120×10⁴ Dalton) will be destroyed at the present of H₂O₂ and CuCl₂ (see, for example, Carbohyr Res. 1999, 321: 228-34; Carbohyr Res. 2006, 341:639-44; and Pathophysiology. 2003, 9: 215-220). It has been also reported that the hyaluronan in synovial fluid is susceptible to H₂O₂, or may be destroyed by OH⁻ derived from H₂O₂ (see, Inflammation. 1993, 17: 403-15). Therefore, H₂O₂ seems to play a role in varying the structure of hyaluronan.

In the present invention, the effects of hyaluronans with various molecular weights on inhibiting the free radical (H₂O₂) production in vitro and in vivo, and on reducing the oxidative damage in chondrocyte had been investigated. We also evaluated the effectiveness of exogenous hyaluronan on restoring and protecting chondrocytes of aged joint, and/or promoting the proliferation thereof. By the disclosure of this invention, the possible mechanism of hyaluronan action in the cavity of joint is illustrated, and an alternative effect of hyaluronan in the treatment or prevention of arthritis is provided. The anti-H₂O₂ hyaluronan may be used in treating and/or preventing a hyperferremia associated disease or condition, such as hematochromatosis, bronze diabetes, and pigmented cirrhosis. Furthermore, the use of hyaluronan in manufacturing healthy foods for improving the condition of arthritis is provided in an embodiment of this invention.

### SUMMARY OF THE INVENTION

In one aspect of the invention, is provided the use of hyaluronan in protecting chondrocytes against damages caused by reactive oxygen.

In another aspect of the invention, is provided the use of hyaluronan in promoting proliferation of chondrocytes. In one embodiment, the proliferation improvement is performed by the regulation of cell cycle in chondrocytes. In another embodiment, the regulation of cell cycle is associated with the up-regulation of Cyclin B1 (G2/M) expression.

In a further aspect of the invention, is provided a pharmaceutical composition for treating or preventing arthritis, which is characterized by comprising hyaluronan with a molecular weight of 500,000-8,000,000 Dalton as the active ingredient. In one embodiment, the hyaluronan is used in alleviating the oxidative damages in chondrocytes.

In a further aspect of the invention, is provided a healthy food for improving the condition of arthritis, which is characterized by comprising hyaluronan with a molecular weight of 50-800×10⁴ Dalton.

### BRIEF DESCRIPTION OF THE FIGURES

FIG. 1 shows the reduced production of H₂O₂ in synovial fluid treated with hyaluronan of various molecular weights.

FIG. 2 shows the reduced amounts of O_{2 -}, H₂O₂, C-reactive protein (CRP), and haptoglobin contained in the synovial fluid of subject treated with hyaluronan.

FIG. 3A shows that the production of free radical is inhibited at the presence of hyaluronan.

FIG. 3B shows the viability of chondrocytes treated with increasing concentration of H₂O₂ at the absence or presence of hyaluronan (20 μl).

FIG. 4 shows the growth curve of chondrocytes obtained from old patient at the absence or presence of hyaluronan (1 mg/ml).

FIG. 5 shows the cell cycle in chondrocytes obtained from old patient treated with hyaluronan (1 mg/ml) measured by flow cytometery.

FIG. 6 shows the expression of cell cycle proteins D1, cdk4, and cdk6 (which regulating G0/G1 phase); and B1 (which regulating G2/M phase) in chondrocytes from old patient treated with hyaluronan (1 mg/ml) measured by Western blotting.

### DETAILED DESCRIPTION OF THE INVENTION

The surfaces of the two bones at the joint are covered in cartilage. Articular cartilage is multi-layered. A thin superficial layer provides a smooth surface for the two bones to slide against each other. Cartilage is composed of cells called condrocytes which are dispersed in a firm gel-like ground substance, called the matrix. The main purpose of cartilage is to provide a framework upon which bone deposition could begin. Another important purpose of cartilage is to provide smooth surfaces for the movement of articulating bones.

### EXAMPLES

In order to illustrate the invention, the following examples are included. However, it is to be understood that these examples do not limit the invention and are only meant to suggest a method of practicing the invention.

The following examples used hyaluronan prepared by the methods disclosed in Japan patents 1284023 and 1353027 (Publication of Examined Application Number: S60-009042, Title: Separating Method for Acidic Polysaccharides from Connective Tissues; and Patent Number 1353027, Publication of Examined Application Number: S61-021241, Title: Separating Method for Acidic Polysaccharides from Connective Tissues), which concluding: hyaluronan of 6,000,000 Dalton (Synvisc, Biomatrix, USA), of 600,000-1,200,000 Dalton (ARTZDispo, Seikagaku, Japan), of 600,000-1,200,000 Dalton (Hikamilon Dispo, Taisho Pharmaceutical Co., Ltd., Japan), of 600,000-1,200,000 Dalton (Lumisteron Dispo, Nissin, Japan), of 600,000-1,200,000 Dalton (Unihylon Dispo, UJI, Japan), of 500,000-730,000 Dalton (Hyalgan, Fidia, Italy), and of 500,000-730,000 Dalton (Suplasyn, Bioniche, Ireland)

### Example 1

### Hyaluronan Reduced Amounts of H₂O₂ and Inflammatory Factors

For demonstrating the effects of hyaluronan injected into patient's articulation on the production of H₂O₂ in synovial, we firstly drew the synovial from hyaluronan-treated patient and analyzed the content of free radical. The method for free radical analysis followed the method described in American Journal of Transplantation 2005, 5:1194-1203, which is incorporated herein by reference.

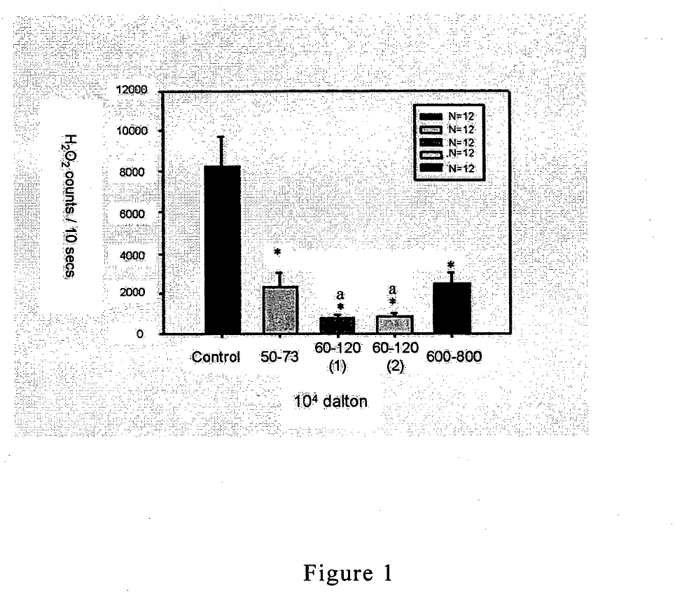

Briefly, the sampled synovial was colded on ice. 200 μl of the cold synovial was placed on iron plate, and then put into the detecting cell of Chemiluminescence detector (CLA-FS1, Tohoku Electronic Ind. Co., Sendai, Japan). After turning on the detector and measuring the backgroung for 50 sec, 500 μl of Luminol (from Sigma Corp., USA, as 0.1 mM solution prepared by disolving the powder in phosphate buffer slution (PBS), stored at 4° C.) or Lucigenin (from Sigma Corp., USA, as 0.1 mM solution prepared by disolving the powder in PBS, stored at 4° C. was added, and then the content of free radicals was measured in 300 sec, with one respective accumulative value obtained at the interval of 10 sec. The background integration in 300 sec was calculated by: (average integration of time-count in 50 sec)×30. The average free radical count for each sample at the interval of 10 sec was calculated as follow: (total area integration under time-count curve−background integration in 300 sec)÷25. As showed in FIG. 1, hyaluronans with molecular weight of 500,000 to 8,000,000 Dalton exhibited an effect on reducing the production of H₂O₂ in synovial fluid. Among the hyaluronans tested in this experiment, the effectiveness thereof in reducing the production of H2O₂ is 600,000-1,200,000 Dalton>500,000-730,000 Dalton>6,000,000 Dalton.

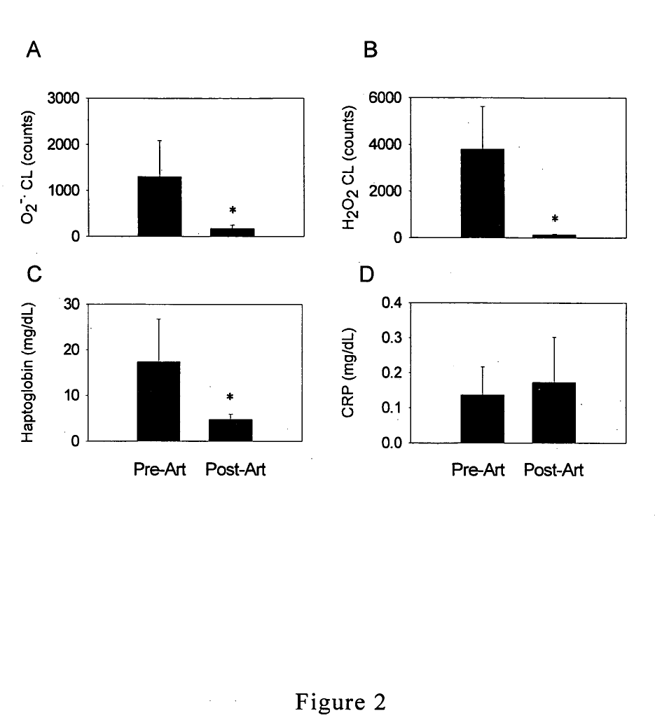

We also used the synovial fluid sampled f synovial fluid from untreated patient as a control. By the results showed in FIG. 2, the content of was significantly reduced in the synovial fluid from the hyaluronan-treated patients as compared to the control synovial fluid, which contained high level of free radicals such as O₂ ^{-.}, H₂O₂.

### Example 2

### The Production of Free Radicals and Mortality of Chondrocytes were deminished by Hyaluronan in Vitro

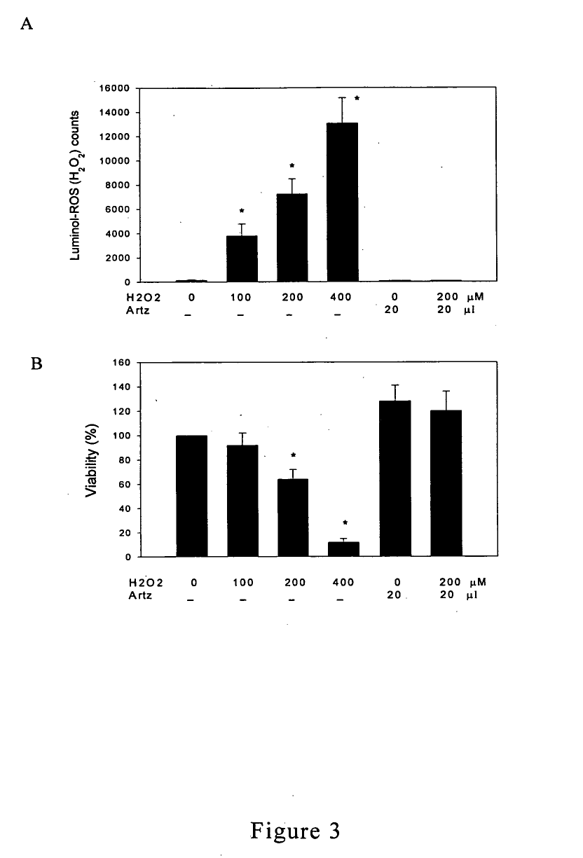

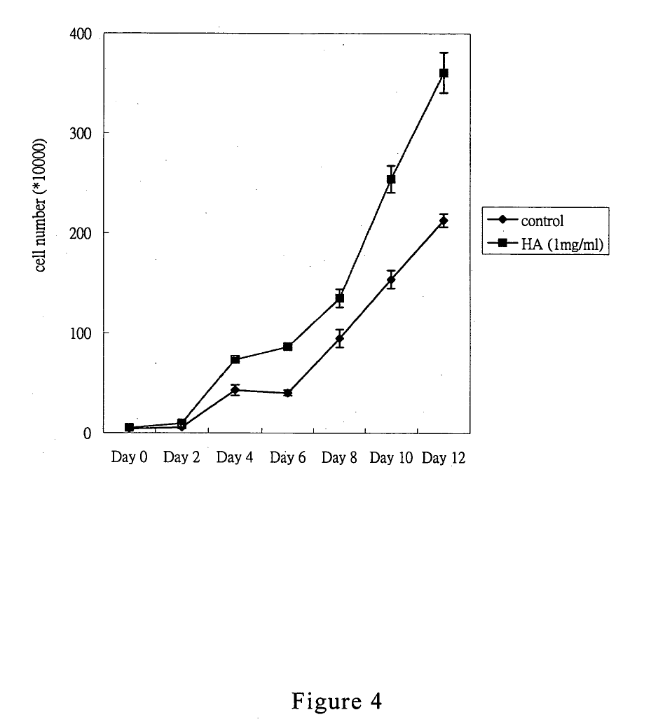

In order to realize the in vitro effects of hyaluronan on the reactivity of free radicals, such as H₂O₂, we took 200 μl of H₂O₂ at various concentrations (0, 100, 200, and 400 μM) for analysing the presence of free radicals by Chemiluminescence detector. Additionally, 20 μl of hyaluronan was provided at the presence of 200 μM H₂O₂. The content of free radical was measured as described above. Results were showed in FIG. 3A. The level of free radical was increased as the increasing concentrations of H₂O₂. However, the production of free radical was inhibited at the presence of hyaluronan (with 200 μM H₂O₂), which was almost at the same level in the H₂O₂-untreated group (i.e., the group at the presence of 0 μM H₂O₂). For knowing that the increased contents of free radical will cause cell death, we also investigated effects of hyaluronan on the mortality caused by free radicals.

The cartilage tissue was collected from a patient older than 60 years old. The primary chondrocytes were isolated and cultured in appropriate medium and conditions described as follow.

The collected cartilage tissue was weighed in a centrifuge tube, and then washed with 10 ml PBS twice. The washed cartilage tissue was transferred onto a 10 cm cell culture dish by a blunt forceps and washed with 10 ml PBS once again. The soft tissue and bone tissue attaching to the cartilage were excised with a scalpel. The separated white cartilage tissue was transferred onto a 10 cm cell culture dish, and then divided into pieces having volume less than 1 mm³ by using a scalpel. To the cell culture dish containing cartilage pieces 60 μl of Typr II cellulose(100mg/ml) was added, and then shaken on a shaker (50 rpm) at 37° C., 5% CO₂ for 4 hr. The digested cartilage tissue was washed with 5 ml of culture medium.

The separated cells were transferred into a sterile 15 ml centrifuge tube, and washed with 10 ml PBS twice. The cells were resuspended in 10 ml of culture medium. 50 μl of the cell culture was sampled for counting viable chondrocytes. 40 μl PBS and 10 μl of 10× Trypan Blue were added to the sampled cell culture, mixed thoroughly and 10 μl of the mixture was dropped into hemocytometer. The cell density was calculated as follow: total counted cell no in 9 lattices X 2×10⁴/9=cell no./ml. Chondrocytes were inoculated into a flask at the density of 1×10⁴/ml, and cultured at 37° C., 5% CO₂, with changing fresh medium every three days. For the displacement of fresh medium, the culture medium was drawn off by a disposable pipette, and 10 ml PBS was carefully added along the wall of flask. After shaking the culture flask gently, PBS was drawn off and 12 ml of fresh medium was added, and then the culture flask was incubated at 37° C., 5% CO₂.

For the in vivo test, various concentrations (0, 100, 200, and 400 μM) of H₂O₂ were added to the cell culture respectively, with the two additional groups added 0 and 200 μM H₂O₂ each plus 20 μl of hyaluronan. The chondrocytes were cultured for one day under the H₂O₂ treatment, and then collected for detecting their viability. As shown in FIG. 3B, the viability of chondrocyte was decreased with the increasing concentrations of H₂O₂. On the contrary, however, the viability of chondrocyte was increased at the presence of hyaluronan. Accordingly, it is suggested that hyaluronan possesses the ability to inhibit the production of free radicals, decrease the mortality of chondrocytes, and further improve the proliferation of chondrocytes.

### Example 3

### Hyaluronan Promoted the Growth of Chondrocytes

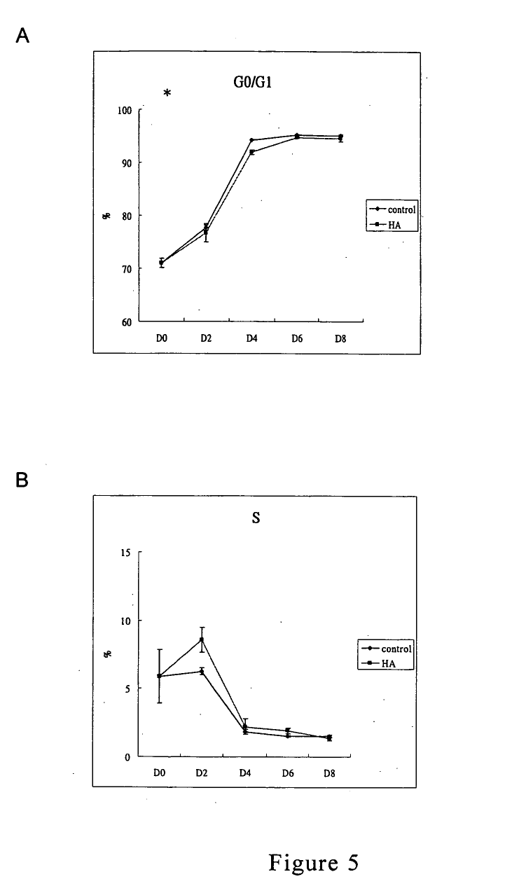

This experiment is provided to evaluate the effects of hyaluronan on the growth of chondrocytes in old patient's joint. The chondrocytes from old patient were isolated and cultured as described in Example 2. Chondrocytes were cultured to 80% confluence and plated in 60 mm dish (each contained about 8×10⁴ cells), then further cultured over night. The culture medium was replaced with fresh medium containing 1 mg/ml hyaluronan (HA group) or fresh medium only (control group), and the cultivation was continued till 12 days, with a replacement of culture medium (with or without hyaluronan) at day 5. The initial cell number was counted at day 0, and taken a count every 2 days. The growth curves of cultured cells were shown in FIG. 4. In day 2 to day 4, the cell number of chondrocytes grew at the presence of hyaluronan (HA group) was significantly greater than control group of about 1.7-fold. The cell number in HA group was greater than control group of above twofold at day 6.

### Example 4

### Hyaluronan Promoted the Growth of Chondrocytes By Controlling Their Cell Cycle

The chondrocytes from old patient were isolated and cultured as described in Example 2. Chondrocytes were cultured to 80% confluence and plated in 100 mm dish (each contained about 3×10⁵ cells). The culture medium was replaced with medium containing 1 mg/ml hyaluronan (HA group) or fresh medium (control group) on next day, and the cultivation was continued till 8 days. Cell sampling was begun at day 0 and kept at intervals of 2 days. The harvested cells were treated as described below for the analysis of cell cycle by a flow cytometery.

The cultured chondrocytes were washed with PBS, trypsinized and suspended in 5 ml of culture medium containing 5% fetal calf serum (FCS). The cells were washed with 5 ml cold PBS, and then fixed in 1 ml of 70% alcohol at −20° C. for more than 1 hr. The fixed cells were washed with 5 ml PBS and spun at low speed (1200 rpm/min), then stained with 1 ml of propidium iodide (PI)/Triton X-100 solution (which containg 0.1% Triton X-100, 0.2 mg/ml RNase A, and 20 μg/ml PI at final concentration, respectively) at room temperature in the dark for 30 min. The sample was mixed throughly and filtered through 35-μm nylon membrane before run on the flow cytometery (FL2-A) for detecting the fluorescence expressed on cells.

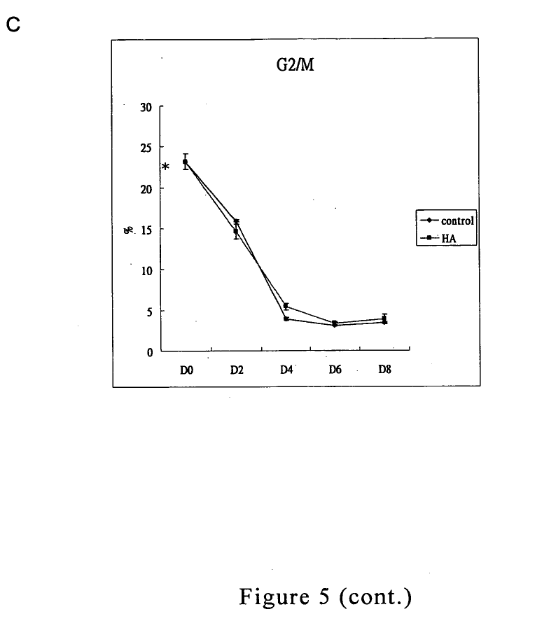

The results were shown in FIG. 5. Referring to the detection of G0/G1 phase in cell cycle (FIG. 5A), the % of cells in G0/G1 phase in the control group was higher than those in HA group at Day 4, otherwise, there is no significant difference between those two groups. By the results in S phase detection (FIG. 5B), there is no statistical difference between HA and control groups from Day 0 to Day 8. On the other hand, it was found that the % of cells in G2/M phase in the HA group was much higher than those in control group at Day 4, but showed no difference after Day 6 (see, FIG. 5C).

### Example 5

### Hyaluronan Promoted the Growth of Chondrocyte By Up-Regulating the Expression of Cyclin B1 (G2/M)

For further understanding the mechanism of hyaluronan action in modulating the cell cycle of chondrocyte, we investigated the expression of various cell cycle regulating factors at the prescence of hyaluronan (1 mg/ml). The chondrocytes cultured and treated as described in Example 2 were harvested at Day 2, 4, 6, and 8 and prepared for Western blotting as follow.

The harvested cells were washed with 1× PBS three times, and RIPA buffer (containing 50 mM Tris (pH7.5), 150 mM NaCl, 1% NP40, 0.1% SDS, 0.5% sodium deoxycholic acid, and proteinase inhibitor) was added to lyse cell and release proteins. The cell lysate was collected in an eppendorf tube and spun at 12,000 rpm, at 4° C. for 30 min. The quantified supernatant containing 20 μg protein was mixed with sampling buffer (with glycerin) at the rate of 1:4, and boiled in 100° C. water bath for 5 min. The prepared protein sample was loaded on SDS-polyacrylamide gel for electrophoresis, and then transferred onto a nylon paper. The nylon paper was blocked with 5% skimed milk in Tris buffer at room temperature for 1 hr. The nylon paper was washed with Tris buffer three times, each for 5 min. The primary antibody was added and the mixture was shaked at room temperature for 1 hr or at 4° C. over night. The nylon paper was washed with Tris buffer five times, each for 5 min. The secondary antibody was added and the mixture was shaked at room temperature for 40 min. The nylon paper was washed with Tris buffer as described above. The Chemiluminescence detecting kit (ECL kit) was added and reacted at room temperature for 1 min, and then developed in dark room.

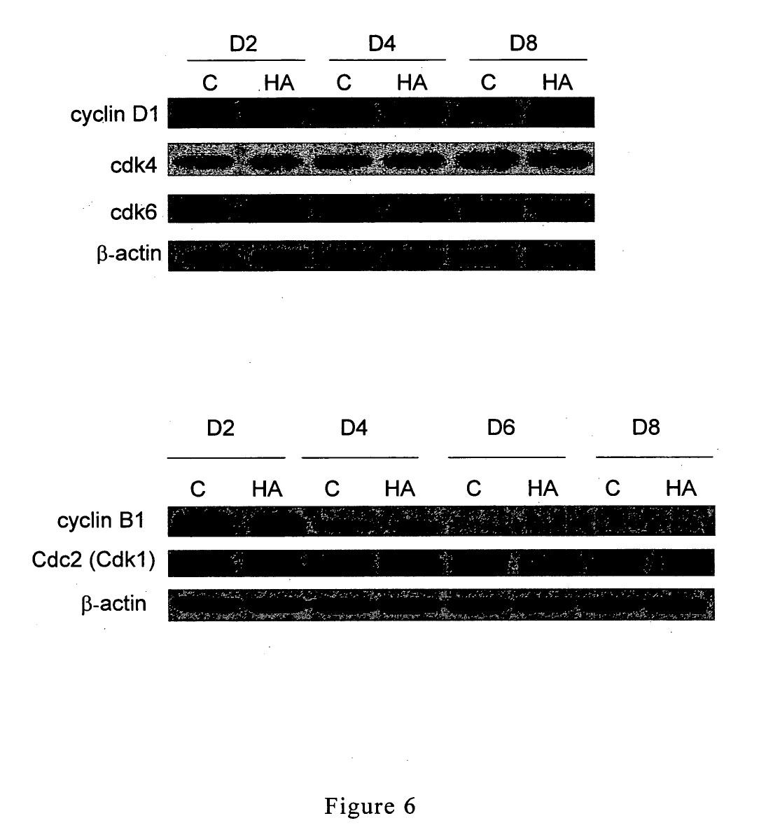

As shown in FIG. 6, the expression of G0/G1 phase regulating proteins, such as Cyclin D1, cdk4, and cdk6, exhibited no difference between the control and HA groups. However, the G2/M phase regulating protein, Cyclin B1, exhibited higher expression level in HA-treated cells than those in control group. Furthermore, the expression of Cyclin B13-associated protein cdc2 was unchanged.

The present invention has been illustrated by the embodiments and examples described above. The skilled in the art will appreciate that any modification or change can be made as if not depart from the spirit and scope of the invention. The present invention was encompassed in the appended claims.

1. A use of hyaluronan in protecting chondrocytes against oxidative damages, wherein the damages are caused by reactive oxygen.

2. The use of claim 1, wherein the molecular weight of the hyaluronan is in the range of 500,000 to 8,000,000 Dalton.

3. The use of claim 2, wherein the molecular weight of the hyaluronan is in the range of 600,000 to 1,200,000 Dalton.

4. The use of claim 1, wherein the molecular weight of the reactive oxygen is selected from the group of OH⁻, H₂O₂, and HOCl.

5. The use of claim 1, wherein the protection of chondrocytes from oxidative damage is achieved by reducing the reactivity of free radicals.

6. The use of claim 1, wherein the hyaluronan is further used in reducing the mortality of chondrocyte.

7. A use of hyaluronan in promoting the proliferation of chondrocytes.

8. The use of claim 7, wherein the promotion in chondrocyte proliferation is achieved by controlling the cell cycle of chondrocytes.

9. The use of claim 8, wherein the hyaluronan is used in the up-regulation of Cyclin B1 (G2/M).

10. A medicament for treating or preventing arthritis, which comprising the hyaluronan, wherein the molecular weight of the hyaluronan is in the range of 500,000 to 8,000,000 Dalton.

11. The medicament of claim 10, wherein the molecular weight of the hyaluronan is in the range of 600,000 to 1,200,000 Dalton.

12. The medicament of claim 10, wherein the use of hyaluronan is for protecting the chondrocytes from oxidative damage.

13. The medicament of claim 10, wherein the use of hyaluronan is for reducing the reactivity of free radicals.

14. A food for improving the condition of arthritis, which comprising the hyaluronan, wherein the molecular weight of the hyaluronan is in the range of 500,000 to 8,000,000 Dalton.

15. The food of claim 14, wherein the molecular weight of the hyaluronan is in the range of 600,000 to 1,200,000 Dalton.
